(19) United States
(12) Patent Application Publication
(10) Pub. No.: US 2025/0384989 A1
(43) Pub. Date: Dec. 18, 2025

(54) SYSTEM AND METHOD FOR PRECISION AND PERSONALIZED NEUROREHABILITATION USING STRATIFIED DATA-DRIVEN DECISION SUPPORT

(71) Applicant: PRS Neurosciences & Mechatronics Research Institute Private Limited, Bangalore (IN)

(72) Inventors: Sharan Srinivasan, Bangalore (IN); Prathiba Sharan, Bangalore (IN); Stafford Michahial, Bangalore (IN); Nikhil. C.H., Bangalore (IN); Sapna K., Bangalore (IN)

(73) Assignee: PRS Neurosciences & Mechatronics Research Institute Private Limited, Bangalore, KA (IN)

(21) Appl. No.: 19/240,206

(22) Filed: Jun. 17, 2025

(30) Foreign Application Priority Data
Jun. 17, 2024 (IN) 202441038858

(51) Int. Cl. G16H 20/70; G16H 10/60; G16H 50/70

(52) U.S. Cl. CPC G16H 20/70; G16H 10/60; G16H 50/70

(57) ABSTRACT

The present invention relates to a cognitive computing-assisted clinical decision support system designed to enable personalized neurological rehabilitation. The system acquires structured user data across clinical, anatomical, radiological, etiological, pathological, and rehabilitation domains to create individualized profiles. These profiles are mapped against a repository of historical cases using analog matching and similarity scoring to generate stratified, evidence-based rehabilitation recommendations. Real-time monitoring of rehabilitation progress is performed using global recovery and function outcome indicators, allowing for dynamic adjustment of treatment plans. Clinician intervention modules ensure safety, interpretability, and context-aware customization. The system incorporates a continuous feedback mechanism to refine future predictions and recommendations, making it increasingly adaptive over time. The invention improves rehabilitation outcome prediction accuracy, reduces recovery variability, and optimizes functional outcomes by transforming static rehabilitation models into intelligent, responsive, and personalized care pathways.

### TECHNICAL FIELD OF THE INVENTION

The present invention relates to the field of functional neurosciences. More specifically, the invention relates to a system and method for evidence-based, stratified, cognitive computing-assisted plans for delivering precision and personalized plans, maximizing outcomes, anticipating/preventing/minimizing complications and their sequelae and optimizing care in neuro rehabilitation and other settings or domains like cardiology, orthopedic etc.

### BACKGROUND OF THE INVENTION

Neurological rehabilitation is currently limited by the absence of a unified, intelligent & intuitive framework that can systematically organize and interpret complex, multidimensional user data in a clinically actionable way. Existing rehabilitation practices rely heavily on fragmented medical records, generalized diagnostic coding, and the subjective experience of multi-disciplinary clinicians who work in silos with no comprehensive and effective trans-disciplinary interactions and hence no combined decisions. This results in anecdotal, individual experience-driven, non-standardized, individualistic rehabilitation protocols that do not adequately reflect the individual's needs, comorbidities, or the maximum possible dynamic recovery potential of users. Additionally, the lack of integrated predictive analytics, realistic progress mapping, real-time feedback loops, and analog-based personalization inhibits timely and appropriate adaptation of rehabilitation plans. As a consequence of this lack of evidence-based science, treatment outcomes are often unpredictable, recovery timelines and hence costs vary widely, and healthcare resources are inefficiently utilized.

Specifically in the field of neurorehabilitation, a critical disconnect exists between the acute care teams—such as neurologists, critical-care physicians, intensivists and neurosurgeons—and the long-term rehabilitation providers, including physiatrists and multidisciplinary therapists. High-quality and structured user data are non-standardized, disjointed, far and few. User data spanning clinical, anatomical, radiological, etiological, pathological, and rehabilitation domains is scattered across vertical, siloed systems, hindering longitudinal user understanding, inter-silo data manipulation, and collaborative care planning. Without a structured method (using a framework) for comparing a user's condition to historically similar cases, clinicians (especially the younger, less experienced) are unable to formulate confidently, precision and personalized rehabilitation strategies or accurately prognosticate or predict recovery outcomes. Moreover, the inability to dynamically revise rehabilitation plans based on real-time progress (or the lack of it) often leads to therapeutic delays or inaccuracies, reduced functional gains, and preventable complications, sometimes even resulting in unexpected mortality or morbidity. Hence, there is a need for a system that enables stratified, data-driven, intuitive, and continuously adaptive rehabilitation planning through integrated user profiling, analog outcome referencing, and real-time rehabilitation optimization.

### OBJECT OF THE INVENTION

A principal object of the invention is to develop a system and method for a ‘clinical decision support/optimization’ engine that enables stratified, cognitive computing modelling for precision & personalized neurological rehabilitation planning, prognosis prediction, progress mapping, and real-time rehabilitation monitoring, with need-based course corrections, based on structured multi-domain user data.

Another object of the invention is to provide a comprehensive, modular system that captures and organizes user-specific clinical, anatomical, radiological, etiological, pathological, and rehabilitation data in a standardized format to generate individualized user profiles for analog case matching and evidence-driven rehabilitation recommendations and prognostication.

Another object of the invention is to enable accurate and dynamic rehabilitation precision & personalization through integration of analog user mapping, similarity scoring, cohort stratification, and predictive analytics modules that utilize historical recovery data to improve prognostic accuracy, map progress (or the lack of it) and optimize rehabilitation strategies (or even course correction).

Another object of the invention is to facilitate continuous monitoring and adaptive rehabilitation plan optimization by incorporating outcome tracking mechanisms, complication analysis engines, and clinician oversight modules, thereby ensuring real-time alignment of rehabilitation interventions with user progress, safety considerations, and functional recovery goals.

These and other objects and characteristics of the present invention will become apparent from further disclosure in the detailed description given below.

### SUMMARY OF THE INVENTION

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

The present invention provides an evidence-based, cognitive computing-assisted ‘clinical decision support system’ for precision & personalized neurological rehabilitation planning, prognosis prediction, and adaptive rehabilitation optimization. Designed to overcome the limitations of generic treatment protocols, silo-based approaches of individual clinicians, anecdotal treatment strategies and fragmented data systems, the invention leverages structured, multi-domain user data to generate individualized rehabilitation recommendations based on real-world recovery evidence. The system integrates modular components including a data acquisition unit, similarity mapping engine, rehabilitation recommendation module, prognosis prediction engine, and outcome monitoring interface to deliver stratified, evidence-based, data-driven rehabilitation strategies. It facilitates dynamic plan adjustments based on user progress, enabling real-time alignment of rehabilitation with evolving clinical needs. The system is suitable for deployment in hospitals, specialized neurorehabilitation centers, and community-based care settings where scalable, personalized, and evidence-backed rehabilitation planning is critical.

In some embodiments, the dynamic rehabilitation system addresses shortcomings of existing approaches such as subjective outcome prediction, lack of dynamic plan revisions, and poor coordination between acute care and rehabilitation teams. By combining analog user case referencing, similarity scoring, cohort-based personalization, and risk-benefit analysis, the system ensures both precision and safety in rehabilitation recommendations, all the while keeping evidence-based data in the forefront. In certain configurations, it incorporates clinician-in-the-loop interventions, adaptive thresholding for plan reassessment, and archival complication databases to guide preventive strategy integration. These evidence-based features enhance clinical reliability and operational adaptability while maintaining a high standard of precision & personalized care across varied user profiles and care environments.

Embodiments of the invention may further provide a method for implementing the dynamic rehabilitation system to deliver real-time, individualized care. This evidence-based method involves acquiring domain-specific user data, generating a multi-domain user profile, and mapping it against a repository of historical cases using a ‘similarity scoring’ engine. Based on the matched analog cases and predicted recovery outcomes, a personalized rehabilitation strategy is recommended and monitored throughout the execution journey. The system continuously evaluates user progress using functional status improvement indicators and either automatically triggers plan modifications if recovery deviates from the expected trajectory or refers to a clinician for an evaluation. These real-time adjustments, supported by continuous feedback to the system's knowledge base, ensure that rehabilitation remains optimized throughout the rehabilitation journey, thereby improving both clinical outcomes and resource efficiency.

To the accomplishment of the foregoing and related ends, one or more aspects comprise the features hereinafter fully described and particularly pointed out in the claims. The following description and the drawings set forth in detail certain illustrative features of one or more aspects. These features are indicative, however, of a few of the various ways in which the principles of various aspects may be employed, and this description is intended to include all such aspects and their equivalents.

### BRIEF DESCRIPTION OF DRAWINGS

The foregoing and other features of the embodiments will become more apparent from the following detailed description of the embodiments when read in conjunction with the accompanying drawings. In the drawings, like reference numerals refer to like elements.

In the following description, numerous specific details are set forth in order to provide a thorough understanding of the embodiments of the invention. It is apparent, however, to one skilled in the art that the embodiments of the invention may be practiced without these specific details or with an equivalent arrangement. In other instances, well-known structures and devices are shown in block diagram form in order to avoid unnecessarily obscuring the embodiments of the invention.

FIG. 1A illustrates a network environment for a dynamic rehabilitation system, according to one embodiment of the invention.

FIG. 1B illustrates a system block diagram of the dynamic rehabilitation system, according to one embodiment of the invention.

FIG. 2 illustrates a generic block diagram of the dynamic rehabilitation system, according to one embodiment of the invention.

FIGS. 3A-3E illustrates specific block diagrams of the dynamic rehabilitation system, according to one embodiment of the invention.

FIG. 4 illustrates a flowchart for implementing the dynamic rehabilitation system, according to one embodiment of the invention.

FIG. 5 illustrates a graph representing recovery curve comparison, according to one embodiment of the invention.

FIG. 6A illustrates a graph representing rehabilitation time saving comparison, according to one embodiment of the invention.

FIG. 6B illustrates a graph representing rehabilitation time saving comparison for the staff, according to one embodiment of the invention.

FIG. 7A illustrates a graph representing deviation from the expected recovery curve, according to one embodiment of the invention.

FIG. 7B illustrates a graph representing deviation from expected recovery curve while using the dynamic system, according to one embodiment of the invention.

### DETAILED DESCRIPTION OF THE INVENTION

Reference will now be made in detail to the description of the present subject matter, one or more examples of which are shown in figures. Each example is provided to explain the subject matter and not as a limitation. Various changes and modifications obvious to one skilled in the art to which the invention pertains are deemed to be within the spirit, scope, and contemplation of the invention.

The embodiments herein and the various features and advantageous details thereof are explained more fully with reference to the non-limiting embodiments that are illustrated in the accompanying drawings and/or detailed in the following description. Descriptions of well-known components and processing techniques are omitted so as not to unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearance of the phrase “in an embodiment” in various places in the specification does not necessarily all refer to the same embodiment, nor are separate or alternative embodiments mutually exclusive of other embodiments. Moreover, various features are described which may be exhibited by some embodiments and not by others. Similarly, various requirements are described, which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon the present disclosure.

As used in this application, the term “Neurorehabilitation” refers to the multidisciplinary process of restoring function and independence in individuals affected by neurological conditions such as stroke, traumatic brain injury, or spinal cord injury. Within the context of this invention, neurorehabilitation is enhanced through personalized, data-driven rehabilitation planning, prognosis forecasting, and adaptive intervention strategies generated using cognitive computing-assisted systems.

The term “Personalized Medicine” refers to tailoring medical treatment to the individual characteristics of each user. In this invention, personalized medicine is operationalized through the use of structured user profiles encompassing clinical, anatomical, radiological, etiological, pathological, and rehabilitation data to generate individualized rehabilitation strategies and predicted recovery timelines.

The phrase “cognitive computing in healthcare” refers to the integration of artificial intelligence algorithms in clinical workflows to support decision-making, diagnosis, and treatment planning. the present invention leverages cognitive computing to analyze user-specific data, identify similar historical cases, and recommend optimized rehabilitation plans based on learned recovery patterns.

As used herein, the term “Clinical Decision Support System” refers to a technology platform that aids healthcare providers in making evidence-based clinical decisions. In the described invention, the decision support system generates personalized rehabilitation plans, predicts functional outcomes, and provides real-time recommendations using analog case data and similarity analysis.

The term “User Stratification” refers to the classification of users into subgroups based on shared clinical or functional characteristics to guide rehabilitation planning. In this invention, user stratification enables scalable mass personalization by forming cohorts from multi-domain profile data and applying optimized rehabilitation templates accordingly.

The phrase “Prognosis Prediction” refers to the estimation of expected health outcomes based on current and historical user data. In the invention, prognosis prediction is performed using structured user inputs, functional assessment markers, and analog recovery outcomes to forecast potential functional gains, timeframes, and probability of success.

The term “Rehabilitation Monitoring” refers to the continuous tracking and evaluation of a user's progress during rehabilitation. In the present invention, rehabilitation monitoring involves the use of functional status improvement indicators to compare actual recovery against expected trajectories and enable timely adjustment of rehabilitation plans.

The term “Analog User Mapping” refers to the process of identifying and referencing historical user cases with similar profiles to inform the care of a current user. The invention applies analog mapping using a similarity scoring engine to align present cases with past recovery patterns, thus enhancing confidence in rehabilitation selection and outcome prediction.

As used in this application, the phrase “Functional Recovery Optimization” refers to strategies aimed at maximizing a user's rehabilitation gains in the shortest feasible timeframe. The invention achieves functional recovery optimization by selecting high-impact therapies based on analog evidence and continuously adapting plans to align with real-time user response.

The term “Multidomain User Profiling” refers to the compilation of user information across various clinical domains to build a comprehensive therapeutic overview. In this invention, multidomain profiling is foundational to rehabilitation personalization, enabling the system to capture and organize key functional, pathological, and treatment-response attributes for each user.

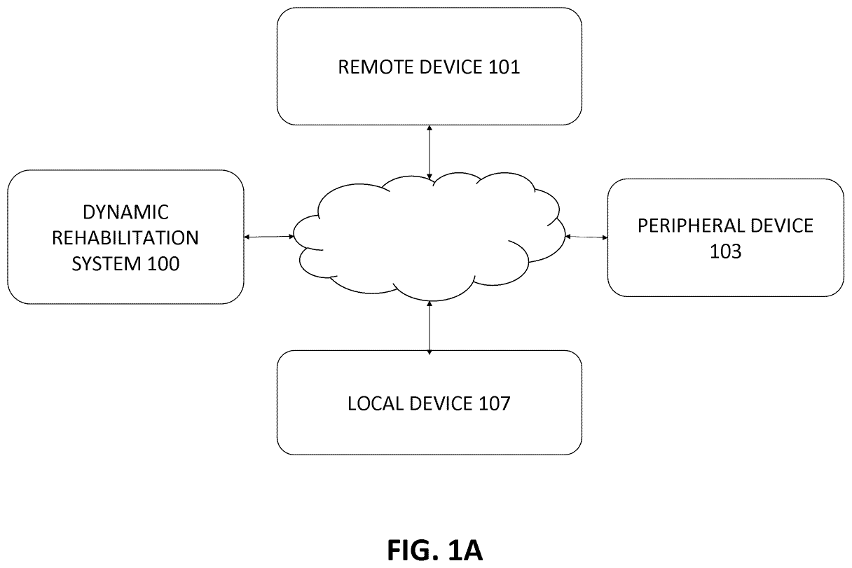

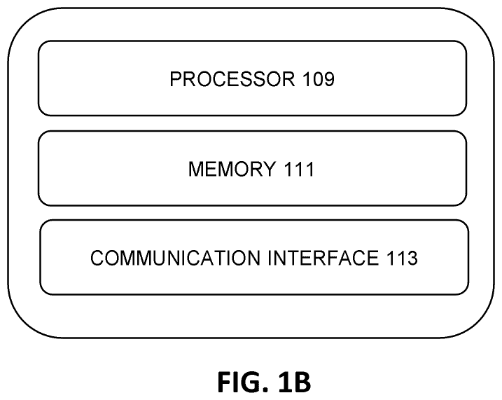

FIG. 1A illustrates a network environment for a dynamic rehabilitation system, according to one embodiment of the invention. In one example embodiment, the network 105 serves as the central communication infrastructure, enabling seamless data exchange among the various components of the dynamic rehabilitation system 100, including modules responsible for rehabilitation planning, outcome prediction, monitoring, and clinician interaction.

In certain embodiments, the dynamic rehabilitation system 100 may be connected to the network 105 to facilitate real-time access to user-specific data, automated updates to rehabilitation recommendation algorithms, and secure logging of rehabilitation outcomes. This connectivity supports cloud-based data storage, remote supervision by clinical teams, and ongoing refinement of predictive models using updated recovery data. The network 105 may also interface with one or more remote devices 101, peripheral devices 103, and local devices 107 to enhance functionality and system interoperability.

According to one example embodiment, the remote device 101 may be connected to the network 105 to allow clinicians, therapists, or administrators to access the dynamic rehabilitation system 100 from remote locations. Via secure mobile or web interfaces, users can monitor ongoing rehabilitation progress, receive outcome predictions, adjust rehabilitation parameters, and configure alert thresholds based on individualized user data. The remote device 101 may also support updates to decision thresholds, similarity mapping parameters, or risk stratification criteria as new cases are added to the system.

In some embodiments, the peripheral device 103 connected to the network 105 may provide extended support to the dynamic rehabilitation system 100. Such a device may include cloud-hosted repositories for user cohort data, hospital EMR integrations, or access to medical knowledge bases that contextualize rehabilitation recommendations. Integration with peripheral devices 103 enables functions like longitudinal recovery tracking, advanced analytics for cohort optimization, and system-wide reporting for research or audit purposes.

In accordance with one embodiment, the local device 107 connected to the network 105 may function as the primary control and monitoring interface for the dynamic rehabilitation system 100. It may be used on-site by healthcare professionals to display real-time rehabilitation recommendations, analog case matches, outcome probabilities, and ongoing user metrics. The local device 107 enables hands-on interaction with system outputs, allowing clinicians to validate or refine generated plans and input real-time responses, ensuring accurate, adaptive, and context-aware rehabilitation delivery.

FIG. 1B illustrates a system block diagram of the dynamic rehabilitation system, according to one embodiment of the invention. According to an example embodiment, FIG. 1B illustrates a system block diagram having a processor 109 at the core of the system. The processor may execute instructions and perform calculations necessary for various tasks.

In one example embodiment, a memory 111 that is connected to the processor 109 stores data and instructions that the processor may need to perform tasks. The memory may include a volatile memory such as RAM, that is used for temporary data storage and also a non-volatile memory such as flash storage, that retains data even when the device is powered off.

In one example embodiment, a communication interface 113 may enable the device to connect and communicate with other devices or networks. The communication interface may include various communication protocols such as Wi-Fi, Bluetooth, or cellular networks that allow the system to send and receive data, updates, and commands.

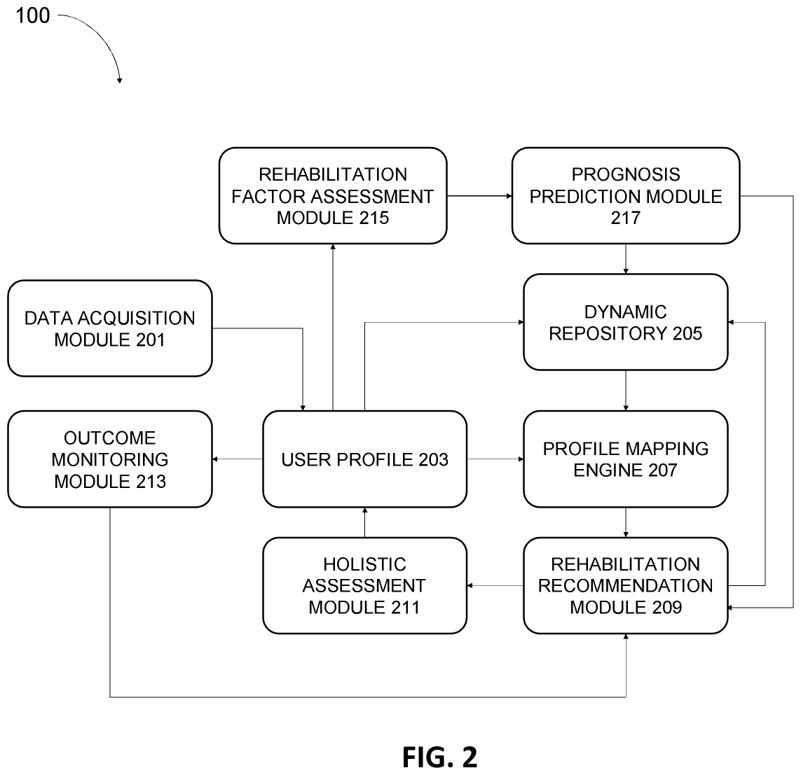

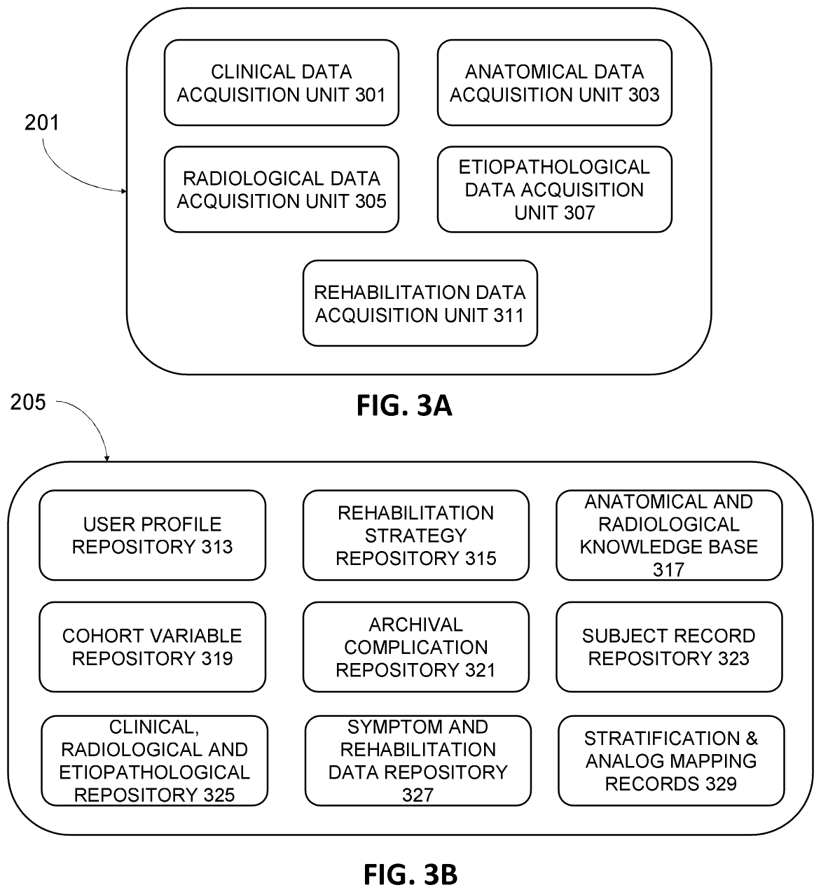

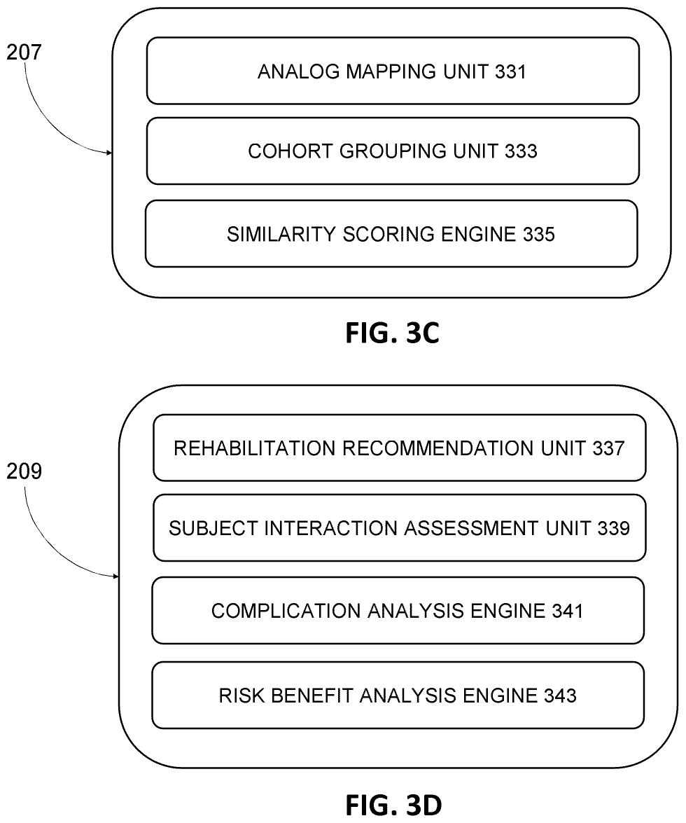

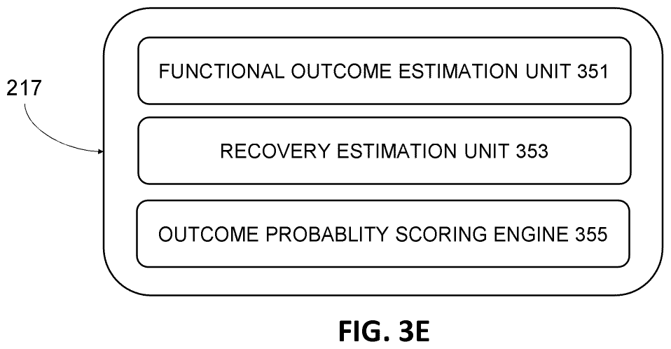

FIG. 2 illustrates a generic block diagram of the dynamic rehabilitation system, according to one embodiment of the invention. According to an example embodiment, FIGS. 3A-3E illustrates specific block diagrams of the dynamic rehabilitation system, according to one embodiment of the invention.

According to an example embodiment, FIG. 2 illustrates generic block diagram of the dynamic rehabilitation system 100 comprising multiple functional modules that work together to deliver personalized rehabilitation planning, outcome prediction, and real-time monitoring. The system 100 operates by acquiring, analyzing, and dynamically updating user-specific rehabilitation data, enabling a stratified and adaptive approach to neurological recovery.

According to an example embodiment, the system 100 includes a data acquisition module 201 configured to collect structured user data across six domains: clinical, anatomical, radiological, etiological, pathological, and rehabilitation. This data is aggregated to generate a user profile 203, which serves as central repository of user-specific attributes and medical history required for personalized rehabilitation planning.

According to an example embodiment, FIG. 3A illustrates a detailed view of the data acquisition module 201 of the dynamic rehabilitation system 100. The data acquisition module 201 is responsible for capturing a comprehensive range of user-specific data across multiple medical domains necessary for generating a structured and holistic user profile 203. Each sub-unit within the module 201 is designed to collect a particular category of data relevant to rehabilitation personalization and outcome prediction.

According to an example embodiment, a data acquisition module 201 comprises a clinical data acquisition unit 301 configured to gather information related to the user's clinical history, diagnoses, neurological symptoms at ictus, and primary or secondary conditions recorded during the acute phase of care. This data is primarily obtained from electronic medical records, discharge summaries, and detailed intake assessment of the patient and is essential for understanding the initial therapeutic context and comorbidity landscape of the user.

According to an example embodiment, an anatomical data acquisition unit 303 collects information regarding the specific brain regions and neural pathways affected by the injury or disease. This data may be sourced from preexisting literature evidences based on brain mapping studies using advanced imaging techniques and existing radiological repositories (i.e. CT, MRI, or PET, fMRI, TMS etc) and helps create a hypothesis of anatomical correlates to functional deficits.

According to an example embodiment, a radiological data acquisition unit 305 is configured to extract structured findings from CT, MRI, or PET scans. This includes lesion characteristics, volumetric measurements, and radiographic biomarkers.

Radiological data confirms the anatomical hypothesis with visual evidence, which is later used by modules such as the prognosis prediction module 217 and profile mapping engine 207. Radiological data also provides additional information on any mild symptoms that could be affecting the patient's function, based on the anatomical knowledge that could have been missed during clinical profiling, to give a more accurate user profile 203.

According to an example embodiment, an etiopathological data acquisition unit 307 captures the root cause and disease mechanism underlying the user's condition, such as ischemic stroke, hemorrhagic injury, traumatic brain injury, tumor-related effects, or degenerative conditions. Understanding the etiology allows the rehabilitation system 100 to stratify users based on causality, thereby enabling more accurate analog matching and risk prediction.

According to an example embodiment, the etiopathological data acquisition unit 307 further collects laboratory findings, histopathology reports, biomarker analysis, and other disease-related parameters that provide insight into disease severity, progression, and systemic implications. This data supports the rehabilitation factor assessment module 215 in identifying clinical red flags and contraindications to certain interventions.

According to an example embodiment, a rehabilitation data acquisition unit 311 is responsible for capturing prior and ongoing rehabilitation strategies used along with corresponding patient response data, functional assessments, and progress notes from various therapists including physical rehabilitation, occupational rehabilitation, speech-language pathology, and cognitive rehabilitation. This unit ensures that real-time and retrospective rehabilitation performance data is included in the user profile 203, allowing for continuous monitoring by the outcome monitoring module 213 and fine-tuning by the rehabilitation recommendation module 209.

According to an example embodiment, the user profile 203 is processed by a rehabilitation factor assessment module 215, which identifies key rehabilitation-relevant attributes such as neurological deficits, rehabilitation responsiveness, and comorbid conditions. These factors are forwarded to a prognosis prediction module 217, which utilizes predictive models to estimate the user's expected functional outcomes, timeline to recovery, and probability of reaching therapeutic milestones.

According to an example embodiment, the system 100 comprises a dynamic repository 205 that stores the data acquired by module 201 and the predictions generated by module 217. The dynamic repository 205 serves as a continuously evolving knowledge base that includes historical user outcomes, rehabilitation protocols, and analog recovery data. This repository is accessed by a profile mapping engine 207, which analyzes the user profile 203 and compares it with previously stored profiles using similarity scoring algorithms.

According to an example embodiment, FIG. 3B illustrates the structure of the dynamic repository 205 of the dynamic rehabilitation system 100. The dynamic repository 205 serves as an evolving knowledge base that aggregates and stores structured data, predictive outcomes, rehabilitation plans, analog recovery records, and clinical knowledge essential for enabling personalized rehabilitation recommendation and real-time rehabilitation monitoring. The repository 205 provides data access and storage capabilities to various system modules including the user profile 203, the profile mapping engine 207, the rehabilitation recommendation module 209, and the prognosis prediction module 217.

According to an example embodiment, a user profile repository 313 stores structured user profiles created from data acquired by the data acquisition module 201. These profiles contain user-specific information across clinical, anatomical, radiological, etiological, pathological, and rehabilitation domains. The repository 313 enables rapid retrieval of user data for similarity mapping, outcome prediction, and rehabilitation personalization.

According to an example embodiment, a rehabilitation strategy repository 315 maintains a catalogue of rehabilitation protocols, intervention plans, and strategy variants collected from historical cases and validated clinical practices. This repository serves as a reference for the rehabilitation recommendation module 209 when generating personalized rehabilitation plans and allows reuse or adaptation of proven strategies for similarly profiled users.

According to an example embodiment, an anatomical and radiological knowledge base 317 stores structured representations of brain anatomy, functional maps, lesion localizations, and associated recovery potentials. This knowledge base supports the prognosis prediction module 217 and the rehabilitation factor assessment module 215 in assessing likely functional impairments and recovery outcomes based on affected brain regions.

According to an example embodiment, a cohort variable repository 319 maintains aggregated variables used for stratifying users into cohorts. These variables may include age group, etiology, severity score, comorbidity index, education, socioeconomic factors, geographical/ethnic background or baseline functional assessment. The repository 319 allows the system 100 to form dynamic user cohorts for scalable mass personalization, accessed by the profile mapping engine 207.

According to an example embodiment, an archival complication repository 321 stores data related to rehabilitation exclusions, risk flags, or clinical complications such as Deep Vein Thrombosis (DVT) development, Pressure sores, musculoskeletal issues including pain, nutrition related issues etc observed in historical users. This repository provides potential prospective risk-awareness to the rehabilitation recommendation module 209, particularly through the complication analysis engine and risk benefit analysis engine, ensuring that contraindicated interventions, high risk interventions or ineffective interventions are filtered out. This helps in prevention of potential complications, early interventions where appropriate and provide a comprehensive care rather than only treating symptomatically.

According to an example embodiment, a subject record repository 323 stores anonymized records of historical user cases used as analog references for similarity matching. These records contain recovery trajectories, rehabilitation responses, complications, and outcomes, and are used by the profile mapping engine 207 to identify the most relevant analogs for a given user profile 203.

According to an example embodiment, a clinical, radiological and etiopathological repository 325 organizes detailed radiology findings and etiological classifications with corresponding clinical presentations to support anatomical and diagnostic comparisons during similarity scoring. It enables the mapping of visual and causative data from historical records to current profiles, thus improving analog matching precision.

According to an example embodiment, a symptom and rehabilitation data repository 327 stores structured data on functional impairments, symptom progression, and rehabilitation effectiveness. It is referenced by both the outcome monitoring module 213 and the rehabilitation recommendation module 209 to assess historical rehabilitation outcomes in similar cases and recommend effective interventions.

According to an example embodiment, a stratification and analog mapping records 329 maintain logs of cohort definitions, similarity match results, and mapping criteria used in prior sessions. This record allows retrospective validation of cohort formation logic and provides traceability for how rehabilitation recommendations were generated based on analog recovery patterns.

According to an example embodiment, the profile mapping engine 207 identifies one or more historically similar cases, which are then utilized by a rehabilitation recommendation module 209 to formulate a personalized rehabilitation strategy. The recommendation module 209 integrates the similarity results from module 207, the prognostic data from module 217, and expert input from a holistic assessment module 211. The holistic assessment module 211 provides a clinician-facing interface that enables medical professionals to review, modify, and validate the recommended rehabilitation strategy, ensuring that human judgment remains central to user care.

According to an example embodiment, FIG. 3C illustrates the internal structure of the profile mapping engine 207 of the dynamic rehabilitation system 100. The profile mapping engine 207 is responsible for identifying historically similar user cases by comparing a newly generated user profile 203 against archived subject profiles stored in the dynamic repository 205. This mapping process plays a critical role in enabling analog recovery-based personalization of rehabilitation strategies.

According to an example embodiment, the profile mapping engine 207 includes an analog mapping unit 331 configured to search the subject record repository 323 and the user profile repository 313 within the dynamic repository 205. The analog mapping unit 331 identifies previously recorded user cases that share structural and functional similarities with the current user profile 203. These analogs are used to infer recovery patterns, rehabilitation responsiveness, and potential complications that may influence rehabilitation planning for the current user.

According to an example embodiment, the profile mapping engine 207 includes a cohort grouping unit 333 that stratifies identified analog cases into meaningful subgroups based on shared attributes drawn from the cohort variable repository 319. These attributes may include primary diagnosis, lesion location, baseline severity, age, comorbidities, or functional status at admission. By forming matched cohorts, the system 100 enables scalable and context-aware rehabilitation recommendation through the rehabilitation recommendation module 209.

According to an example embodiment, the profile mapping engine 207 includes a similarity scoring engine 335 that computes similarity scores between the current user profile 203 and the stored analog profiles. This scoring is based on weighted multidomain feature comparisons using clinical, anatomical, radiological, etiological, pathological, and rehabilitation parameters. The similarity scoring engine 335 prioritizes cases that closely resemble the user in critical outcome-determining dimensions, thereby enhancing the accuracy of rehabilitation selection and prognosis estimation by the rehabilitation recommendation module 209 and the prognosis prediction module 217.

According to an example embodiment, the outputs of the profile mapping engine 207—including matched cases, cohort groupings, and similarity scores—are transmitted to the rehabilitation recommendation module 209 to assist in selecting the most appropriate rehabilitation strategy. Additionally, these outputs are stored in the stratification and analog mapping records 329 of the dynamic repository 205 to maintain transparency, traceability, and consistency in rehabilitation personalization logic.

According to an example embodiment, FIG. 3D illustrates the detailed structure of the rehabilitation recommendation module 209 of the dynamic rehabilitation system 100. The rehabilitation recommendation module 209 is responsible for generating and dynamically updating a personalized rehabilitation strategy for a user based on input from multiple upstream modules, including the profile mapping engine 207, prognosis prediction module 217, holistic assessment module 211, and relevant data from the dynamic repository 205.

According to an example embodiment, the rehabilitation recommendation module 209 comprises a rehabilitation recommendation unit 337 configured to synthesize personalized rehabilitation plans by evaluating the matched analog cases from the analog mapping unit 331, similarity scores from the similarity scoring engine 335, and cohort insights from the cohort grouping unit 333. The recommendation unit 337 selects rehabilitation strategies from the rehabilitation strategy repository 315 based on historical outcomes and real-world evidence to generate a user-specific rehabilitation plan aimed at optimizing functional recovery within the predicted timeline.

According to an example embodiment, the rehabilitation recommendation module 209 includes a subject interaction assessment unit 339, which analyzes potential interactions between different elements of the rehabilitation strategy, pharmacological treatment strategies like potential drug-drug interactions and the unique characteristics of the user profile 203. This unit accounts for factors such as ongoing treatments, medication regimes, rehabilitation overlap, and interdependencies between interventions. It ensures that the selected rehabilitation strategies do not conflict with each other or with the user's current health status, comorbidities, or functional limitations.

According to an example embodiment, the rehabilitation recommendation module 209 includes a complication analysis engine 341 that references historical complication data stored in the archival complication repository 321. This engine identifies any risks or adverse events associated with similar rehabilitation strategies in matched analog cases. Based on this analysis, the system may automatically exclude or flag high-risk interventions, prompting clinician review via the holistic assessment module 211 before finalizing the rehabilitation plan.

According to an example embodiment, the rehabilitation recommendation module 209 includes a risk benefit analysis engine 343 configured to evaluate the trade-offs between potential functional gains and associated risks for each rehabilitation strategy under consideration. The engine compares benefit projections from the prognosis prediction module 217 against risk metrics from the complication analysis engine 341, ensuring that the recommended strategy aligns with the user's risk profile, recovery goals, and overall treatment feasibility.

According to an example embodiment, finalized rehabilitation strategy is implemented and monitored through an outcome monitoring module 213, which tracks user progress in real time. If the monitored outcomes deviate from predicted trajectories, module 213 communicates with the recommendation module 209 to update and refine the rehabilitation strategy dynamically, enabling closed-loop, adaptive rehabilitation management.

According to an example embodiment, FIG. 3E illustrates the structure of the prognosis prediction module 217 of the dynamic rehabilitation system 100. The prognosis prediction module 217 is designed to forecast a user's likely recovery trajectory and rehabilitation responsiveness based on structured user data acquired via the data acquisition module 201 and consolidated within the user profile 203. This module provides foundational inputs to the rehabilitation recommendation module 209 and supports dynamic updates to rehabilitation plans by predicting functional outcomes, timelines, and confidence levels.

According to an example embodiment, the prognosis prediction module 217 includes a functional outcome estimation unit 351 configured to determine the maximum level of functional recovery a user is likely to achieve. This unit utilizes historical analog data retrieved from the subject record repository 323, anatomical insights from the anatomical and radiological knowledge base 317, and baseline status data from the user profile 203 to forecast long-term capabilities across mobility, cognition, speech, or activities of daily living.

According to an example embodiment, the prognosis prediction module 217 includes a recovery estimation unit 353 that estimates the time duration required for the user to reach the predicted functional level. This estimate may be expressed in days, weeks, or months and is based on prior case timelines from the stratification and analog mapping records 329. The unit 353 helps set realistic expectations for rehabilitation planning and resource allocation by forecasting how soon critical milestones-such as independent ambulation or task re-engagement-can be achieved or in deteriorating cases, where palliative care or death is predicticted, reallocation of resources to help optimize resource utilization.

According to an example embodiment, the prognosis prediction module 217 includes an outcome probability scoring engine 355, which calculates a confidence score for each predicted outcome based on statistical match strength, historical outcome variance, and data completeness of the user profile 203. This probability score helps clinicians and the recommendation module 209 assess the reliability of the forecast and informs the degree of caution or flexibility to be exercised when implementing the suggested rehabilitation plan.

According to an example embodiment, the outputs from the functional outcome estimation unit 351, recovery estimation unit 353, and outcome probability scoring engine 355 are transmitted to the dynamic repository 205 for ongoing reference. These outputs are also directly consumed by the rehabilitation recommendation module 209 to guide rehabilitation personalization and risk management.

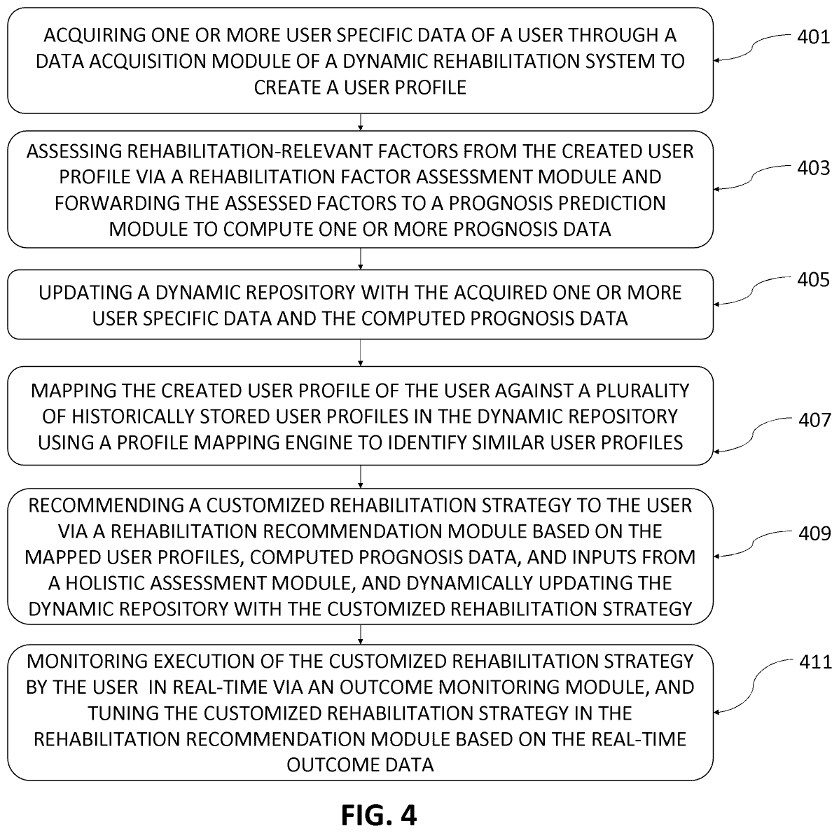

FIG. 4 illustrates a flowchart for implementing the dynamic rehabilitation system, according to one embodiment of the invention. At step 401, the method includes acquiring one or more user specific data of a user through a data acquisition module of a dynamic rehabilitation system to create a user profile. At step 403, the method includes assessing rehabilitation-relevant factors from the created user profile via a rehabilitation factor assessment module and forwarding the assessed factors to a prognosis prediction module to compute one or more prognosis data. At step 405, the method includes updating a dynamic repository with the acquired one or more user specific data and the computed prognosis data. At step 407, the method includes mapping the created user profile of the user against a plurality of historically stored user profiles in the dynamic repository using a profile mapping engine to identify similar user profiles. At step 409, the method includes recommending a customized rehabilitation strategy to the user via a rehabilitation recommendation module based on the mapped user profiles, computed prognosis data, and inputs from a holistic assessment module, and dynamically updating the dynamic repository with the customized rehabilitation strategy. At step 411, the method includes monitoring execution of the customized rehabilitation strategy by the user in real-time via an outcome monitoring module, and tuning the customized rehabilitation strategy in the rehabilitation recommendation module based on the real-time outcome data.

According to an example embodiment, at step 401, one or more user-specific data points are acquired through the data acquisition module 201 of the dynamic rehabilitation system 100. This data includes clinical, anatomical, radiological, etiological, pathological, and rehabilitation-related parameters, collected via respective acquisition units 301 through 311. The acquired data is processed to generate a user profile 203, representing a multidimensional summary of the user's present condition and medical history. This profile forms the foundation for personalized rehabilitation planning.

According to an example embodiment, at step 403, the rehabilitation factor assessment module 215 evaluates the user profile 203 to extract key rehabilitation-relevant factors such as severity of deficits, presence of comorbidities, and responsiveness to prior interventions. These factors are forwarded to the prognosis prediction module 217, which computes one or more personalized recovery predictions. The module utilizes the functional outcome estimation unit 351, recovery estimation unit 353, and outcome probability scoring engine 355 to determine the anticipated functional outcomes, estimated recovery timelines, and likelihood scores for achieving specified milestones.

According to an example embodiment, at step 405, the dynamic repository 205 is updated with both the acquired user-specific data and the prognosis results. The repository comprises multiple sub-repositories such as the user profile repository 313, rehabilitation strategy repository 315, and clinical, radiological and etiopathological repository 325. This structured storage ensures that accurate and context-rich data is made available for retrieval by other modules for analog referencing and rehabilitation strategy generation.

According to an example embodiment, at step 407, the profile mapping engine 207 compares the user profile 203 against previously stored subject records in the dynamic repository 205. The analog mapping unit 331 identifies historically similar cases, while the cohort grouping unit 333 organizes them into cohorts based on common attributes derived from the cohort variable repository 319. The similarity scoring engine 335 assigns quantitative match scores, enabling prioritization of analog cases for reference during rehabilitation recommendation.

According to an example embodiment, at step 409, a personalized rehabilitation strategy is generated by the rehabilitation recommendation module 209. The rehabilitation recommendation unit 337 selects rehabilitation plans based on analog case outcomes and stored strategies. The subject interaction assessment unit 339 evaluates interdependencies between rehabilitation components and user-specific constraints. The complication analysis engine 341 checks for contraindications using historical data from the archival complication repository 321, while the risk benefit analysis engine 343 weighs potential functional gains against identified risks. The holistic assessment module 211 enables clinicians to validate or modify the generated plan before finalization. The customized plan is then stored in the dynamic repository 205 for execution and monitoring.

According to an example embodiment, at step 411, the execution of the rehabilitation strategy is continuously monitored in real time using the outcome monitoring module 213. These units assess whether the user's functional recovery aligns with expected outcomes. If deviations or regressions are detected, the outcome monitoring module 213 communicates the findings to the rehabilitation recommendation module 209, which updates the rehabilitation strategy accordingly. This enables the system 100 to adapt the rehabilitation plan in response to real-world performance, maintaining relevance and therapeutic precision throughout the recovery process.

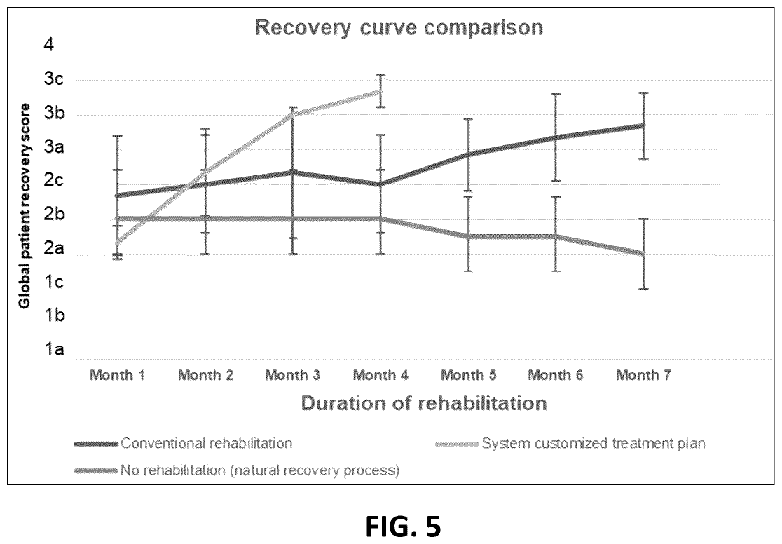

FIG. 5 illustrates a graph representing recovery curve comparison, according to one embodiment of the invention. According to an example embodiment, the graph depicting recovery curve comparison illustrates the difference in functional improvement between neurologically disabled people getting no rehabilitation to conventional rehabilitation methods and those guided by the dynamic rehabilitation system 100. Over a 12-week period, users undergoing rehabilitation through conventional approaches show a gradual and modest increase in Coma to Community (C2C) scores. In contrast, those managed using the dynamic rehabilitation system 100 demonstrate earlier and more sustained improvements in functional recovery. The system's use of analog user mapping, prognostic forecasting, and personalized rehabilitation recommendations enables users to reach higher C2C scores within shorter timeframes. This embodiment demonstrates the system's effectiveness in accelerating rehabilitation outcomes and providing structured, individualized care that adapts dynamically to the user's progress.

The Coma to Community (C2C) score is a clinical measure used to assess the functional recovery of patients who have experienced severe neurological injuries, particularly in cases of coma or brain injury. It is a scale that tracks the patient's progression from a state of coma (or minimal consciousness) to a level where they can reintegrate into their community and daily activities. The score evaluates various functional domains such as mobility, communication, self-care, and social participation.

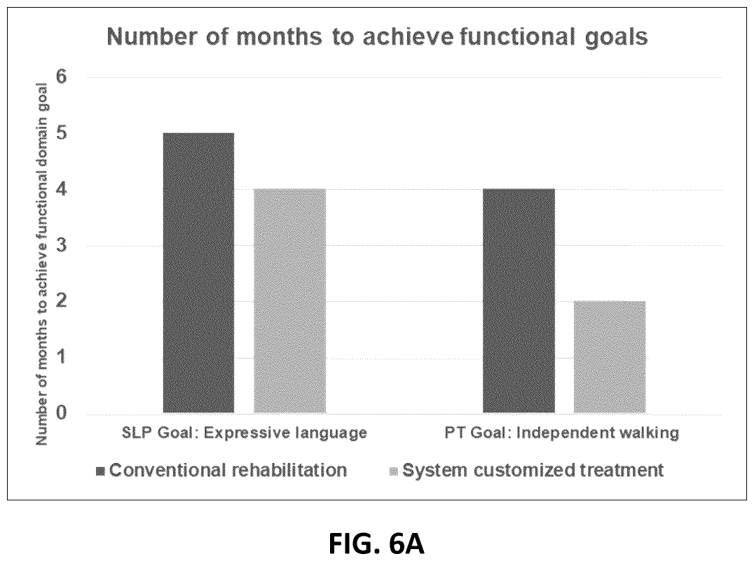

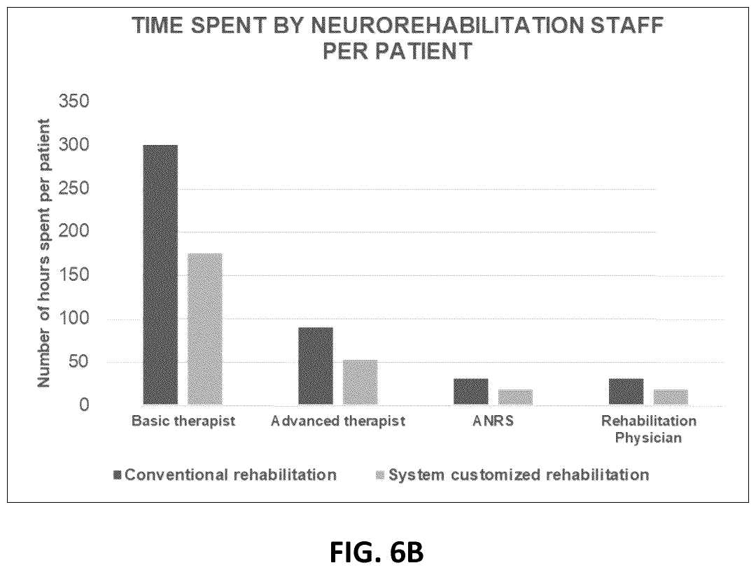

FIG. 6A illustrates a graph representing rehabilitation time saving comparison, according to one embodiment of the invention. According to an example embodiment, the dynamic rehabilitation system 100 demonstrates a significant reduction in the average time required to reach key functional milestones when compared to conventional manual triaging approaches. As illustrated in the rehabilitation time savings graph, users undergoing rehabilitation through conventional rehabilitation typically achieved walking independence in approximately 4 months, and speech or expressive language improvements in 5 months. In contrast, users managed through the dynamic rehabilitation system 100 reached the same milestones notably earlier-within 2 months for walking independence, and 4 months for speech improvements. These improvements are attributed to the system's ability to personalize rehabilitation strategies based on structured user profiling, analog user mapping, and real-time adaptive monitoring, thereby accelerating functional gains and reducing overall rehabilitation timelines.

FIG. 6B illustrates a graph representing rehabilitation time saving comparison for the staff, according to one embodiment of the invention. According to an example embodiment, the dynamic rehabilitation system 100 demonstrates a significant reduction in the average time required to be spent on rehabilitating a patient when compared to conventional approaches. As illustrated in the rehabilitation time savings graph, the time spent on each patient by the care team members at different levels of the hierarchy i.e., basic therapist, advanced therapist, Advanced neurorehabilitation specialists (ANRS) and Rehabilitation physicians, has considerably reduced. Time spent per patient has reduced from 300 hours to 175 hours for basic therapist, 90 to 53 hours for advanced therapists, 30 to 18 hours for ANRS and 30 to 18 hours for rehabilitation physicians.

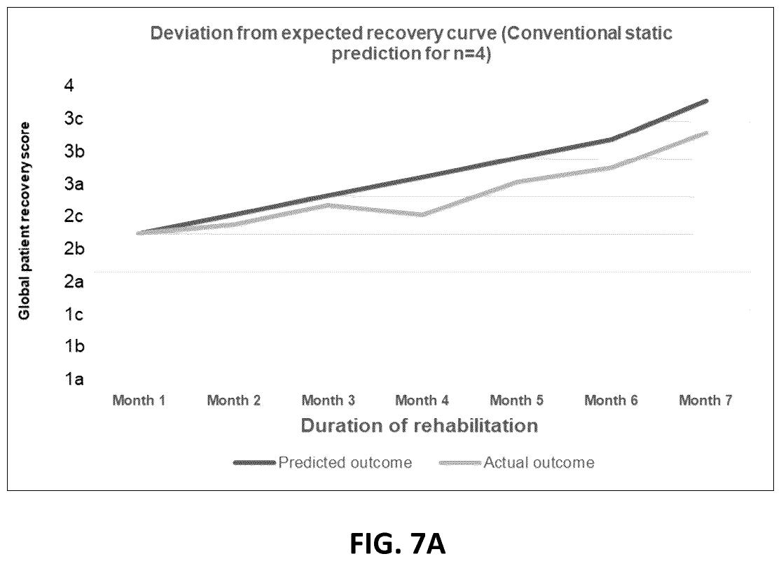

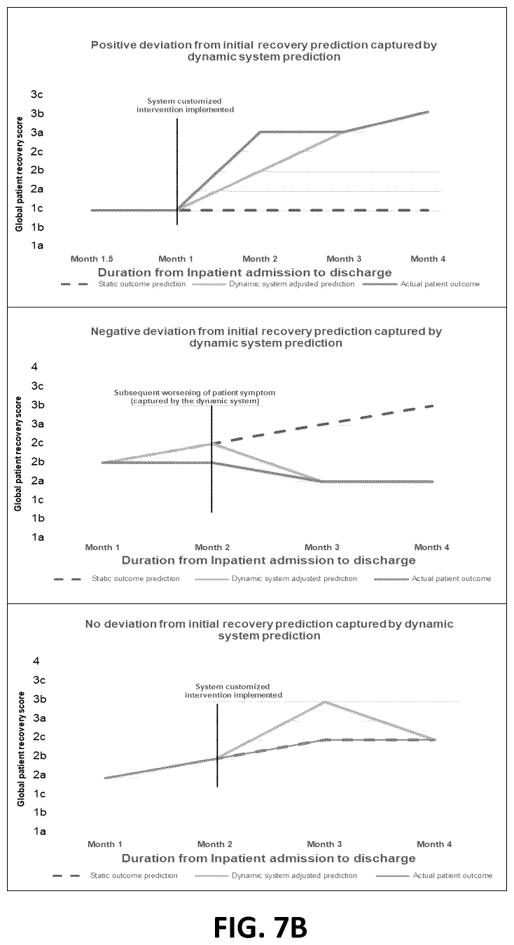

FIG. 7A illustrates a graph representing deviation from expected recovery curve while using the conventional system, according to one embodiment of the invention. According to an example embodiment, the system evaluates predictive reliability by measuring the deviation from expected recovery outcomes. A comparison is conducted between rehabilitation plans generated through conventional manual triaging and those generated by the dynamic rehabilitation system 100. Results show that manual triaging leads to a higher deviation from predicted recovery trajectories, with an average prediction accuracy of 25-50% with an average deviation of one level difference on a global patient recovery scoring scale.

FIG. 7B illustrates a graph representing deviation from expected recovery curve while using the dynamic system, according to one embodiment of the invention. In contrast to the static system cohort-based trajectory adjustments are made by the dynamic system as depicted by the 3 different case scenarios presented. The dynamic rehabilitation system 100 maintains a significantly higher performance accuracy of 50-75%, achieved through its integration of analog user mapping, stratified cohort matching, and real-time outcome monitoring. This increase in performance accuracy demonstrates the system's ability to generate accurate, data-backed predictions and sustain alignment with actual recovery progress, thereby improving rehabilitation reliability and clinical confidence.

TABLE. 1 (as shown below) illustrates a table representing functional outcome gain, according to one embodiment of the invention.

TABLE 1

| | | Dynamic system |
|---|---|---|
| | Conventional | based treatment |
| | (prediction and | modification/ |
| Metric | treatment) | personalization |
| Operational throughput | 40% | 70% |
| (% patients rehabilitated | | |
| in one quarter) | | |
| ICU mortality rates | 48% | 8% |
| Reduction in length of | 21 days | 18 days (>20% |
| stay in ICU | | reduction in |
| | | length of stay) |
| Rehabilitation timeline | 6-9 months (for | 3-6 months (for |
| | segment 2 patients | segment 2 patients |
| | to reach segment 3) | to reach segment 3 - |
| | | Up to 40% reduction |
| | | in time |
| Global patient recovery | 25-50% | 50-75% (25-50% |
| outcome Prediction | | increase in |
| accuracy | | prediction accuracy) |

In one example embodiment, the dynamic system-based treatment modification and personalization model significantly enhances the operational throughput and patient recovery compared to conventional prediction and treatment approaches. The dynamic system is designed to rehabilitate 70% of patients within one quarter, as opposed to the 40% rehabilitation rate achieved by traditional methods. Additionally, the dynamic model demonstrates a dramatic reduction in ICU mortality rates, with only 8% mortality, as compared to the 48% mortality rate observed in conventional systems. This system also reduces the length of stay in ICU by 16 days, which represents a more than 20% reduction in patient recovery time. Rehabilitation timelines are optimized in the dynamic system, requiring only 3-6 months for patients to reach segment 3, compared to the 6-9 months required for segment 2 patients under conventional methods. Furthermore, the dynamic model increases global patient recovery outcome prediction accuracy by up to 75%, offering a 25-50% improvement over traditional systems.

According to an example embodiment, the dynamic rehabilitation system 100 utilizes the data acquisition module 201 to systematically collect multi-domain user data, including clinical, anatomical, radiological, etiological, pathological, and rehabilitation parameters. This structured intake allows the system to construct a detailed user profile, which is then matched against historical cohorts stored in the dynamic repository 205 using the profile mapping engine 207. By identifying analog cases with similar impairments and demographic markers, the system enables the generation of rehabilitation plans that are precisely aligned with the individual's recovery needs. As a result, users benefit from reduced time to achieve critical milestones such as walking independence, speech recovery, and self-care functionality.

According to an example embodiment, the prognosis prediction module 217 receives the analog-matched profiles and projects likely recovery outcomes based on empirical recovery curves observed in previous cases. These predictions are quantified through probability scores and expected timelines, which are compared in real time with the user's actual progress. If a deviation from the expected trajectory is detected, the rehabilitation factor assessment module 215 is triggered to re-estimate the recovery outlook and suggest necessary adjustments. This continuous feedback loop reduces variance from the predicted outcome, thereby enhancing clinician trust in the system's forecasting accuracy.

According to an example embodiment, the rehabilitation recommendation module 209 processes the mapped profile data and prognostic insights to formulate rehabilitation strategies that are both evidence-informed and risk-evaluated. These strategies are validated by the holistic assessment module 211, which incorporates clinician feedback before plan finalization. The integration of clinical judgment ensures safety and appropriateness, while the system's algorithmic rigor ensures precision. This dual-check mechanism results in higher domain-specific outcome gains in areas such as mobility, cognition, communication, and activities of daily living.

According to an example embodiment, the outcome monitoring module 213 continuously evaluates user performance during rehabilitation by comparing it with the predicted recovery curve generated earlier. By tracking changes in Coma to Community (C2C) scores and functional metrics across sessions, the system determines whether the rehabilitation plan remains effective or requires modification. This capacity for dynamic adaptation minimizes the number of sessions required per user and shortens the overall duration of rehabilitation, directly impacting clinical efficiency and user throughput.

According to an example embodiment, the dynamic repository 205 serves not only as a storage unit but also as a continuously evolving knowledge base. With each user case, the repository is updated with new data on rehabilitation inputs, observed outcomes, deviation patterns, and intervention adjustments. This learning capability enables the system to refine future recommendations, making it progressively more accurate and adaptive. Consequently, the number of users rehabilitated per quarter increases, and staff time is utilized more efficiently, validating the operational scalability of the dynamic rehabilitation system 100.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A dynamic rehabilitation system for stratified, personalized rehabilitation planning and recovery optimization, the system comprising:
a) a data acquisition module, wherein the data acquisition module is configured to collect multi-domain user information, including clinical, anatomical, radiological, etiological, pathological, and rehabilitation data via a plurality of data acquisition units;
b) a user profile module, wherein the user profile module is configured to generate a structured user profile based on the collected multi-domain user information;
c) a dynamic repository, wherein the dynamic repository is configured to store rehabilitation strategies, user profiles, analog subject records, symptom data, anatomical and radiological references, contraindication records, and stratification mappings in a plurality of repositories;
d) a profile mapping engine, wherein the profile mapping engine comprises an analog mapping unit, a cohort grouping unit, and a similarity scoring engine, configured to map analog profiles from the dynamic repository with the generated structured user profile;
e) a rehabilitation recommendation module, wherein the rehabilitation recommendation module comprises a rehabilitation recommendation unit, an interaction assessment unit, a complication analysis engine, and a risk-benefit analysis engine;
f) a holistic assessment module, wherein the holistic assessment module is configured to enable clinician validation and user interaction assessment to refine rehabilitation recommendations;
g) an outcome monitoring module, wherein the outcome monitoring module is configured to tracks user progress in real time;
h) a rehabilitation factor assessment module, wherein the rehabilitation factor assessment module is configured to evaluate multi-domain factors affecting recovery of the user to support adaptive decision-making; and
i) a prognosis prediction module, wherein the prognosis prediction module comprises a functional outcome estimation unit, a recovery estimation unit, and an outcome probability scoring engine,
wherein the dynamic rehabilitation system is configured to personalize neurological rehabilitation planning, prognosis prediction, and real-time rehabilitation monitoring based on structured multi-domain user data, matched analog records, and continuous outcome evaluation.

2. The system of claim 1, wherein the plurality of data acquisition units of the data acquisition module comprises a clinical data acquisition unit, an anatomical data acquisition unit, a radiological data acquisition unit, an etiopathological data acquisition unit, and a rehabilitation data acquisition unit.

3. The system of claim 1, wherein the plurality of repositories of the dynamic repository comprises a user profile repository, a rehabilitation strategy repository, an anatomical and radiological knowledge base, a cohort variable repository, an archival complication repository, a subject record repository, a clinical, radiological, and etiopathological repository, a symptom and rehabilitation data repository, and a stratification and analog mapping records.

4. The system of claim 1, wherein the profile mapping engine is configured to compute a similarity score between the structured user profile and analog subject records stored in the dynamic repository using the similarity scoring engine.

5. The system of claim 1, wherein the rehabilitation recommendation module is configured to generate one or more rehabilitation strategies based on the rehabilitation recommendation unit, while dynamically validating treatment feasibility through the subject interaction assessment unit and the complication analysis engine.

6. The system of claim 1, wherein the outcome monitoring module is configured to continuously track user's functional status improvement.

7. The system of claim 1, wherein the prognosis prediction module is configured to estimate functional recovery levels and timeframes of the user using the functional outcome estimation unit and recovery estimation unit, and to assign a confidence level using the outcome probability scoring engine.

8. The system of claim 1, wherein the dynamic rehabilitation system is configured to personalize neurorehabilitation planning, prognosis prediction, and real-time rehabilitation monitoring based on structured multi-domain user data, matched analog records, and continuous outcome evaluation.

9. The system of claim 1, wherein the dynamic repository is configured to store rehabilitation strategies, historical recovery data, and complication reports to support ongoing optimization of rehabilitation strategies.

10. The system of claim 1, wherein the rehabilitation recommendation module automatically adapts the rehabilitation strategies in response to changes in user status and recovery patterns.

11. The system of claim 1, wherein the dynamic rehabilitation system is scalable for deployment in hospital networks, rehabilitation centers, and community care settings.

12. The system of claim 1, wherein the dynamic rehabilitation system is configured to update the dynamic repository with newly acquired user data, historical analog data, and continuously generated rehabilitation strategies.

13. The system of claim 1, wherein the holistic assessment module enables healthcare professionals to provide expert input and validation for the generated rehabilitation strategies.

14. A method for implementing the dynamic rehabilitation system, comprising:
a) acquiring, one or more user specific data of a user, through a data acquisition module of a dynamic rehabilitation system, to create a user profile;
b) assessing, rehabilitation-relevant factors from the created user profile via a rehabilitation factor assessment module, and forwarding the assessed factors to a prognosis prediction module, to compute one or more prognosis data;
c) updating, a dynamic repository with the acquired one or more user specific data and the computed prognosis data;
d) mapping, the created user profile of the user against a plurality of historically stored user profiles in the dynamic repository using a profile mapping engine, to identify similar user profiles;
e) recommending, a customized rehabilitation strategy to the user via a rehabilitation recommendation module, based on the mapped user profiles, computed prognosis data, and inputs from a holistic assessment module, and dynamically updating the dynamic repository with the customized rehabilitation strategy; and
f) monitoring, execution of the customized rehabilitation strategy by the user in real-time via an outcome monitoring module, and tuning the customized rehabilitation strategy in the rehabilitation recommendation module based on the real-time outcome data.

15. The method of claim 14, wherein updating the dynamic repository comprises storing the user profile in the user profile repository, the prognosis data in the stratification and analog mapping records, and synchronizing with previously stored analog records.

16. The method of claim 14, wherein acquiring, one or more user specific data of the user comprises acquiring clinical, anatomical, radiological, etiological, pathological, and rehabilitation-related data from the data acquisition module.

17. The method of claim 14, wherein tuning the customized rehabilitation strategy further comprises re-estimating prognosis using the functional outcome estimation unit, recovery estimation unit, and the outcome probability scoring engine, and revising rehabilitation recommendations accordingly to improve recovery alignment.

18. The method of claim 14, wherein mapping the created user profile of the user against the plurality of historically stored user profiles comprises using the similarity scoring engine and cohort grouping unit of the profile mapping engine.

19. The method of claim 14, wherein recommending the customized rehabilitation strategy comprises evaluating potential interactions between multiple rehabilitation strategies using the interaction assessment unit of the rehabilitation recommendation module.

20. The method of claim 14, wherein monitoring execution of the customized rehabilitation strategy further comprises tracking functional improvement using the outcome monitoring module and dynamically adjusting the strategy based on progress.
